(19) United States
(12) Patent Application Publication
(10) Pub. No.: US 2015/0055091 A1
(43) Pub. Date: Feb. 26, 2015

(54) OPTICAL TOMOGRAPHIC IMAGING APPARATUS

(71) Applicant: CANON KABUSHIKI KAISHA, Tokyo (JP)

(72) Inventor: Tomoyuki Ikegami, Hiratsuka-shi (JP)

(21) Appl. No.: 14/463,050

(22) Filed: Aug. 19, 2014

(30) Foreign Application Priority Data
Aug. 23, 2013 (JP) 2013-173231

(51) Int. Cl. A61B 3/10; A61B 3/14

(52) U.S. Cl. CPC A61B 3/102; A61B 3/1025; A61B 3/14
USPC 351/206

(57) ABSTRACT

An optical tomographic imaging apparatus acquires tomographic images of an object to be examined; where the images are based on multiplexed reference light and return light returned from the object irradiated by measurement light via a scanning unit. The apparatus includes a splitting unit to split light irradiated from a light source into the measurement light and the reference light, and a focusing unit disposed on the optical path of the measurement light between the splitting unit and scanning unit. A primary magnification of the optical system on the optical path of the measurement light or the observation light path, and a numerical aperture of the light source irradiating the measurement light, are configured such that a maximum inclination of light rays included in the light flux of the measurement light is maintained within ±2 degrees of inclination with respect to the optical axis of the optical path.

### BACKGROUND OF THE INVENTION

1. Field of the Invention

The present invention relates to an optical tomographic imaging apparatus used for ophthalmologic diagnosis and treatment and the like.

2. Description of the Related Art

There are currently known various ophthalmologic devices using optical devices. Examples of optical devices for observing an eye to be examined include anterior eye portion photography apparatuses, fundus cameras, confocal scanning laser ophthalmoscopes (SLO), and so forth. Of these, optical tomographic imaging apparatuses using optical coherence tomography (OCT) taking advantage of multi-wavelength light wave interference is an apparatus capable of taking high-resolution tomographic images of samples, and are coming to be indispensable ophthalmologic devices in the retinal outpatient field (hereinafter referred to as “OCT apparatus”).

An OCT apparatus irradiates a sample with measurement light, which is low-coherence light, and performs high-sensitivity measurement by of backscattered light from the sample using an interference system or an interference optical system. A feature of low-coherence light is that high-resolution tomographic images can be obtained by broadening the bandwidth of the wavelength. An OCT apparatus also can scan a sample with measurement light to obtain a high-resolution tomographic image. Accordingly, OCT apparatuses are in widespread used in retinal ophthalmologic diagnosis and so forth, since they can acquire tomographic images of the retina at the fundus of an eye to be examined.

On the other hand, OCT apparatuses serving as ophthalmologic apparatuses usually are provided with an optical system, such as a fundus observation or anterior eye portion observation optical system, to adjust alignment between the apparatus and eye to be examined. OCT apparatuses are used along with these optical systems by using light of different wavelengths for each optical system. The apparatus is configured so that wavelength separation is performed by a wavelength separation unit such as a dichroic mirror or the like. However, OCT apparatuses use low-coherence light with broad bandwidth of the wavelength, so wavelength separation of light of a wavelength used in the fundus observation or anterior eye portion observation optical systems and so forth, and light of a wavelength used in the OCT apparatus, is difficult.

U.S. Pat. No. 5,537,162 describes situating a beam scanner position at a back focal plane of a lens, so that the incident angle of the beam entering the dichroic mirror during beam scanning is constant. This allows the characteristics of the dichroic mirror to be made uniform, and wavelength separation accuracy can be improved.

However, the beam scanner and lens in U.S. Pat. No. 5,537,162 are driven integrally when performing focal adjustment of the fundus of the eye to be examined. A lens having a back focal plane situated at the beam scanner tends to be large in size, in order to obtain scanning light of the beam scanner. Accordingly, the driving mechanism becomes complex since it has to integrally move the beam scanner and the large lens. Further, integrally moving these components means that the measurement light source, which is in a conjugate relationship optically with the fundus position, also needs to be moved at the same time. In a case where the measurement light source is an optical fiber end, the optical fiber must be moved as well, leading to concern that the polarization state may change. Wavelength separation characteristics also change in accordance with the incident angle of light as to the dichroic mirror.

### SUMMARY OF THE INVENTION

It has been found desirable to provide an optical tomographic imaging apparatus with a simplified driving system where a polarization state is maintained without moving the measurement light source, which can prevent deterioration in wavelength separation characteristics at the dichroic mirror.

According to one aspect of the present invention, an optical tomographic imaging apparatus acquires tomographic images of an object to be examined; where the images are based on multiplexed reference light and return light returned from the object to be examined which has been irradiated by measurement light via a first lens. The optical tomographic imaging apparatus includes: a first lens; a scanning unit disposed on the optical path of the measurement light, and configured to scan the object to be examined by the measurement light; a second lens disposed on the optical path of the measurement light between the scanning unit and the first lens; an optical path branching unit disposed between the first lens and the second lens, and configured to branch, from the optical path of the measurement light, an observation optical path to observe the object to be examined; a splitting unit configured to split light irradiated from the light source into the measurement light and the reference light; and a focusing lens disposed on the optical path of the measurement light between the splitting unit and the scanning unit. The second lens and the scanning unit are disposed such that an angle of the measurement light, scanned by the scanning unit, with respect to the optical path branching unit is maintained. On the optical path between the first lens and the second lens, a primary imaging magnification of the optical system on the optical path of the measurement light or the observation light path, and a numerical aperture of the light source irradiating the measurement light, are configured such that a maximum inclination of light rays included in the light flux of the measurement light is maintained within ±2 degrees of inclination with respect to the optical axis of the optical path.

Further features of the present invention will become apparent from the following description of exemplary embodiments with reference to the attached drawings.

### BRIEF DESCRIPTION OF THE DRAWINGS

FIG. 1 is a diagram illustrating a schematic configuration of an optical tomographic imaging apparatus according to a first embodiment.

FIG. 2 is a diagram illustrating a light flux of a pupil of the optical tomographic imaging apparatus according to the first embodiment.

FIG. 3 illustrates an eye to be examined being scanned in the X-direction.

FIG. 4 illustrates an anterior eye portion image, fundus two-dimensional image, and B-scan image, displayed on a monitor.

FIG. 5 is a diagram illustrating a schematic configuration of an optical tomographic imaging apparatus according to a second embodiment.

FIG. 6 is a diagram illustrating a light flux of a pupil of the optical tomographic imaging apparatus according to the second embodiment.

FIG. 7 is a diagram describing divergence of the light flux on the measurement optical path.

FIG. 8 is a diagram illustrating an example of the relationship between imaging refractivity and greatest inclination.

FIGS. 9A and 9B are diagrams illustrating the relationship between transmittance characteristics of a dichroic mirror and incident angle of light rays.

### DESCRIPTION OF THE EMBODIMENTS

Embodiments will be described with reference to the attached drawings. Note that the same configurations are denoted by the same reference numerals through the present Specification.

### First Embodiment

### OCT Optical System

### Apparatus Configuration

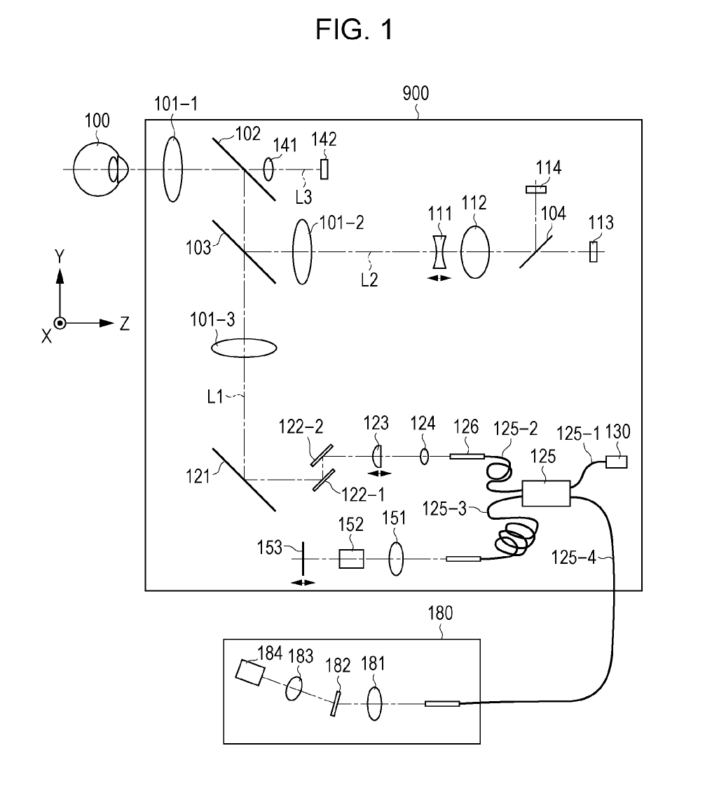

The configuration of an optical tomographic imaging apparatus (OCT apparatus) according to a first embodiment will be described with reference to FIG. 1. The OCT apparatus includes an optical head 900 and a spectroscope 180. The OCT apparatus acquires tomographic images of objects to be examined, based on light obtained by multiplexing return light from the object to be examined which is irradiated with measurement light through a scanning unit, and reference light corresponding to the measurement light.

First, the interior configuration of the optical head 900 will be described. The optical head 900 consists of a measurement optical system for imaging anterior eye portion images, fundus two-dimensional images, and tomographic images, of an eye 100 to be examined. An object lens 101-1 is situated facing the eye 100 to be examined, with the optical path being separated on the optical axis thereof by a first dichroic mirror 102 and a second dichroic mirror 103, which serve as optical path branch portions. That is, the optical path is separated into wavelength band ranges for each of a measurement optical path L1 of the OCT optical system, a fundus observation optical path/fixation lamp optical path L2, and an anterior eye portion observation optical path L3.

The optical path L2 is further branched by wavelength band range into optical paths for a fundus observation charge-coupled device (CCD) 114 and fixation lamp 113 by a third dichroic mirror 104. Now, of lenses 101-2, 111, and 112, the lens 111 is driven by a motor, omitted from illustration, for focal adjustment of the fixation lamp and for fundus observation. The CCD 114 has sensitivity to the wavelength of illumination light for fundus observation, which is omitted from illustration, specifically around 780 nm. On the other hand, the fixation lamp 113 generates visible light so as to prompt fixation of the subject. A lens 141 and infrared CCD 142 for anterior eye portion observation are disposed on the optical path L3. The infrared CCD 142 has sensitivity to the wavelength of illumination light for anterior eye portion observation, which is omitted from illustration, specifically around 970 nm.

The optical path L1 makes up the OCT optical system as described above, and is used to image tomographic images of the fundus of the eye 100 to be examined. More specifically, the optical path L1 is used to acquire interfering signals for forming tomographic images. A lens 101-3, a mirror 121, and a X-scanner 122-1 (first scanning unit) and Y-scanner 122-2 (second scanning unit) which make up a scanning unit. The X-scanner 122-1 and Y-scanner 122-2 scan light on the fundus of the eye 100 to be examined in an X-direction (main scanning direction) which is an example of a first direction, and in a Y-direction (sub-scanning direction) which is an example of a second direction intersecting the first direction. While FIG. 1 illustrates the optical path between the X-scanner 122-1 and Y-scanner 122-2 as being parallel to the plane of the drawing, in reality it is configured perpendicular to the plane of the drawing.

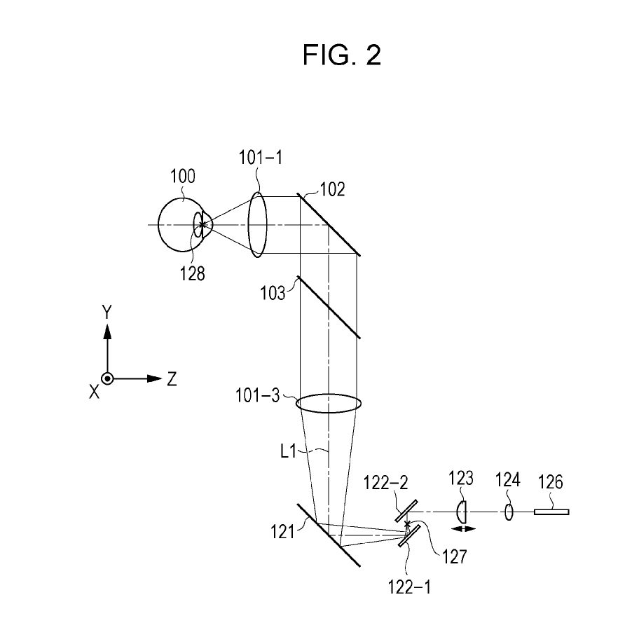

Now, detailed description of configurations on the optical path L1, the conjugate relationship of pupil position with regard to the optical path L1, and the light flux of the pupil will be described with reference to FIG. 2. The configuration is such that a position conjugate with a predetermined member, such as the anterior eye portion of the eye 100 to be examined, or the like, is situated between the first and second scanning units. In the present embodiment, a scanner center position 127 of the X-scanner 122-1 and Y-scanner 122-2 and the pupil position 128 of the eye 100 to be examined are in a conjugate relationship.

The lens 101-1 (first lens), lens 101-3 (second lens), and X-scanner 122-1 and Y-scanner 122-2 (or scanner center position 127) are disposed so that the light flux between the lens 101-1 and lens 101-3 is generally parallel. Due to this configuration, the optical path of which a measurement light deflecting unit is an object point is generally parallel between the lens 101-1 and lens 101-3. Accordingly, the incident angle to the first dichroic mirror 102 and the second dichroic mirror 103 can be made to be the same regardless of having performed scanning at the X-scanner 122-1 and Y-scanner 122-2.

A measurement light source 126 is the light source for the measurement light to be input to the measurement optical path. The measurement light source 126 in the present embodiment is a fiber end, which is in an optically conjugate relationship with the fundus of the eye 100 to be examined. Of lenses 123 and 124, the lens 123 is driven in either direction indicated by arrows in FIG. 2, by a motor omitted from illustration, for focal adjustment. Focal adjustment is performed such that light emitted from the measurement light source 126, which is the fiber end, is imaged on the fundus. The lens 123 serves as a focus adjustment unit, and is situated between the measurement light source 126 and the X-scanner 122-1 and Y-scanner 122-2 which are measurement light deflecting units. Accordingly, the larger lens 101-3 and a fiber 125-2 connected to the measurement light source 126 do not have to be moved. This focus adjustment enables an image of the measurement light source 126 to be focused on the fundus of the eye 100 to be examined, and return light from the fundus of the eye 100 to be examined to be efficiently returned to the fiber 125-2 via the measurement light source 126.

Next, the configuration of the optical path of light emitted from a light source 130, a reference optical system, and the spectroscope 180, illustrated in FIG. 1, will be described. A Michelson interferometer is configured including the light source 130, a mirror 153, a dispersion compensation glass 152, a photocoupler 125, optical fibers 125-1 through 125-4, a lens 151, and the spectroscope 180. The optical fibers 125-1 through 125-4 are single-mode optical fibers integrated by connection at the photocoupler 125.

Light emitted from the light source 130 passes through the optical fiber 125-1, and is split at the photocoupler 125 into measurement light emitted to the optical fiber 125-2 side and reference light emitted to the optical fiber 125-3 side. The measurement light passes through the aforementioned OCT optical system optical path, whereby the fundus of the eye 100 to be examined, which is the observation object, is irradiated. The measurement light reflected and scattered at the retina passes through the same optical path again, and reaches the photocoupler 125.

On the other hand, the reference light passes through the optical fiber 125-3, lens 151, and dispersion compensation glass 152 inserted so that the dispersion of the measurement light and reference light agree, and reaches the mirror 153 where it is reflected. The reflected reference light then passes through the same optical path and reaches the photocoupler 125.

The photocoupler 125 multiplexes the measurement light and reference light, which become interfering light. Interference occurs when the optical path length of the measurement light and the optical path length of the reference light are approximately the same. The position of the mirror 153 in the optical axis direction is adjustably held by a motor and driving mechanism, which are omitted from illustration, so as to be capable of changing the optical path length of the reference light so as to match the optical path length of the measurement light which changes according to the eye 100 to be examined. The interfering light is guided to the spectroscope 180 via the optical fiber 125-4.

The spectroscope 180 includes a lens 181, a diffractive grating 182, a lens 183, and a line sensor 184. Interfering light emitted from the optical fiber 125-4 passes through the lens 181 to become generally parallel light, dispersed by the diffractive grating 182, and imaged on the line sensor 184 by the lens 183.

Next, the light source 130 will be described. The light source 130 is a super luminescent diode (SLD), which is a typical low-coherence light source. The center wavelength is 855 nm, and the wavelength bandwidth is approximately 100 nm. This wavelength bandwidth is an important parameter which affects optical-axis-direction resolution of the obtained tomographic image. While the type of the light source is described as SLD here, any light source which emits low-coherence light may be used, such as amplified spontaneous emission (ASE) or the like. Near-infrared light is suitable for the center wavelength, taking into consideration the fact that an eye to be examined is the object of measurement. The wavelength is preferably as short as possible, since the center wavelength affects the lateral resolution in the obtained tomographic image. Thus, the center wavelength has been set to 855 nm from the above two reasons.

While a Michelson interferometer is used in the present embodiment as the interferometer, a Mach-Zehnder interferometer may be used instead. Preferably, a Mach-Zehnder interferometer is used in a case where the light quantity difference between the measurement light and reference light is great, and a Michelson interferometer is used in a case where the light quantity difference is relatively small.

### Method of Imaging Tomographic Image

The optical tomographic imaging apparatus can image tomographic images of desired portions of the fundus of the eye 100 to be examined by controlling the X-scanner 122-1 and Y-scanner 122-2.

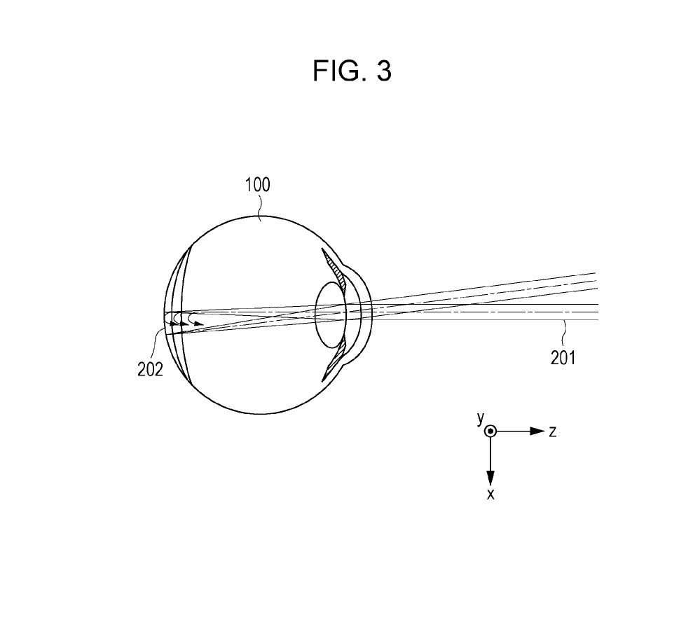

FIG. 3 illustrates the eye 100 to be examined being irradiated with measurement light 201 so that the fundus 202 is scanned in the X-direction. Information of a predetermined number of imaging lines in an X-directional imaging range at the fundus 202 is obtained by the line sensor 184. Luminance distribution obtained at a certain position in the X-direction by the line sensor 184 is subjected to fast Fourier transform (FFT). The linear luminance distribution obtained by FFT is converted into concentration or color information for distribution on a monitor. This is called an “A-scan image”. A two-dimensional image obtained by arraying multiple A-scan images is called a “B-scan image”. After multiple A-scan images have been imaged to construct one B-scan image, the Y-directional scan position is moved an X-directional scanning is performed again, whereby multiple B-scan images can be obtained. Multiple B-scan images, or a three-dimensional tomographic image formed of multiple B-scan images is displayed on the monitor, which the examiner can use for diagnosis of the eye to be examined.

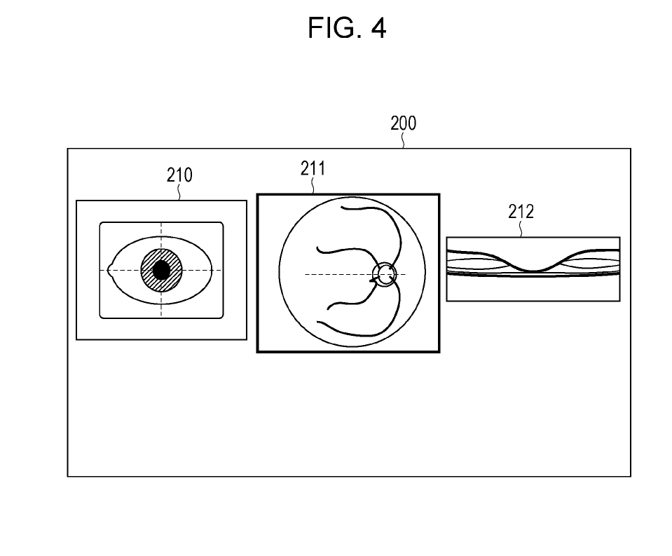

FIG. 4 illustrates an example of an anterior eye portion image 210, a fundus two-dimensional image 211, and a B-scan image 212 which is a tomographic image, displayed on the monitor 200. The anterior eye portion image 210 is an image obtained by processing output of the infrared CCD 142, and displayed. The fundus two-dimensional image 211 is an image obtained by processing output of the CCD 114, and displayed. The B-scan image 212 is an image obtained by processing output of the line sensor 184 as described above, and displayed.

As described above, the optical tomographic imaging apparatus according to the present embodiment has a focus adjustment unit (the lens 123 and an unshown driving mechanism), which performs focal adjustment of the eye to be examined, situated between the measurement light deflecting unit (X-Y scanner) which deflects the measurement light, and the measurement light source 126. A first lens (lens 101-1) and a second lens (lens 101-3) are situated on the measurement optical path between the measurement light deflecting unit (X-Y scanner) and the eye 100 to be examined, and an optical path branch portion (first dichroic mirror 102 and second dichroic mirror 103) is situated between the first lens (lens 101-1) and second lens (lens 101-3).

That is, situating a focus lens between the fiber-side measurement light source and the X-Y scanner serving as a measurement light deflecting unit, does away with the need to move the large-sized lens 101-3, the fiber 125-2 connected to the measurement light source 126, and so forth. Thus, the driving mechanism can be simplified. Further, there is no need to move the fiber end, so an optical tomographic imaging apparatus in which the polarization state is maintained, can be provided.

Moreover, the optical tomographic imaging apparatus according to the present embodiment has the positions of the first lens (lens 101-1) and second lens (lens 101-3), and the measurement light deflecting unit (X-Y scanner) adjusted in their placement, so that the light on the measurement light optical path between the first lens (lens 101-1) and second lens (lens 101-3) is parallel. Accordingly, the incident angle of beams entering the first dichroic mirror 102 and second dichroic mirror 103 can be made constant, thereby improving the wavelength separation accuracy.

While description has been made in the present embodiment with regard to an eye to be examined, scanning may be performed on other objects to be examined, such as skin, internal organs, or other body parts. The present invention is applicable to imaging apparatuses other than ophthalmological apparatuses, such as endoscopes or the like.

### Second Embodiment

### SLO Optical System

### Apparatus Configuration

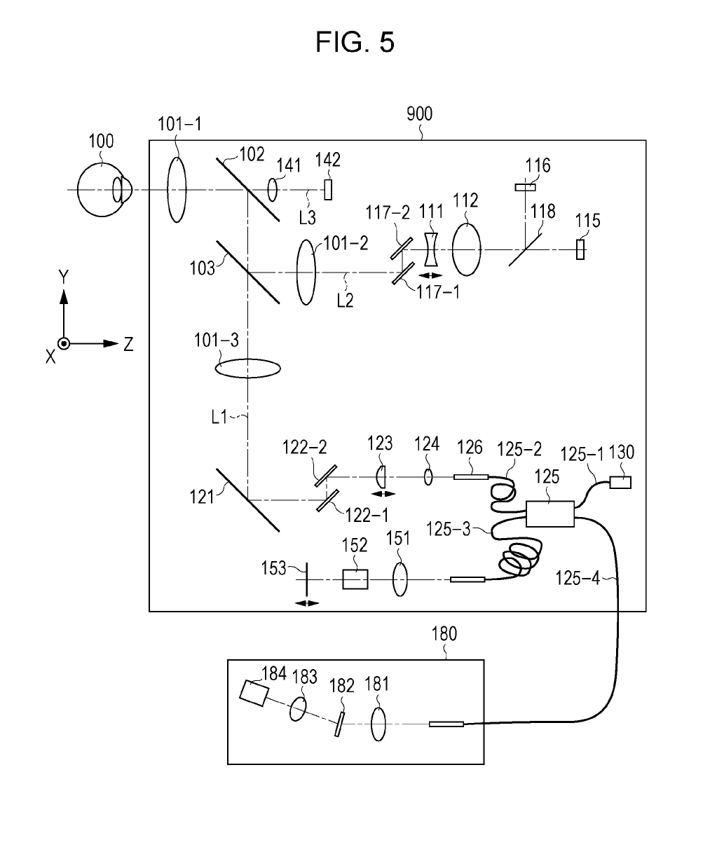

The configuration of an optical tomographic imaging apparatus (OCT apparatus) according to a second embodiment will be described with reference to FIG. 5. The OCT apparatus includes an optical head 900 and spectroscope 180, in the same way as the first embodiment.

In the first embodiment, the optical path L2 has been described as acquiring a fundus two-dimensional image of the eye 100 to be examined by the CCD 114 for fundus observation. Conversely, the second embodiment is configured such that an X-scanner and Y-scanner are disposed on the optical path L2, and a fundus two-dimensional image can be acquired by scanning a spot on the fundus. The configurations of the other optical paths L1 and L3, and the configuration of the spectroscope 180 are the same as described in the first embodiment, so description will be omitted here.

Hereinafter, description will be made primarily regarding the configuration on the optical path L2, which is the portion different from the configuration in the first embodiment. The lenses 101-2, 111, and 112 are illustrated in the same way as in the first embodiment, the lens 111 being driven by a motor omitted from illustration, for focal adjustment to perform fundus observation. The light source 115 generates light having a wavelength of 780 nm. An X-scanner 117-1 (first observation scanning unit) and Y-scanner 117-2 (second observation scanning unit), to irradiate the fundus of the eye 100 to be examined with scan light from a light source 115 for fundus observation, are disposed on the optical path L2. The X-scanner 117-1 and Y-scanner 117-2 function as an observation scanning unit. The lens 101-2 (third lens) is positioned so that the focal position is around the center position of the X-scanner 117-1 and Y-scanner 117-2. The X-scanner 117-1 is configured as a polygon mirror to scan the X-direction at high speed. Alternatively, the X-scanner 117-1 may be configured as a resonating mirror. A single detector 116 consists of an avalanche photodiode (APD), and detects scattered and reflected return light from the fundus. A prism 118 is formed by vapor deposition on a perforated mirror or hollow mirror, and separates illumination light from the light source 115 and return light from the fundus.

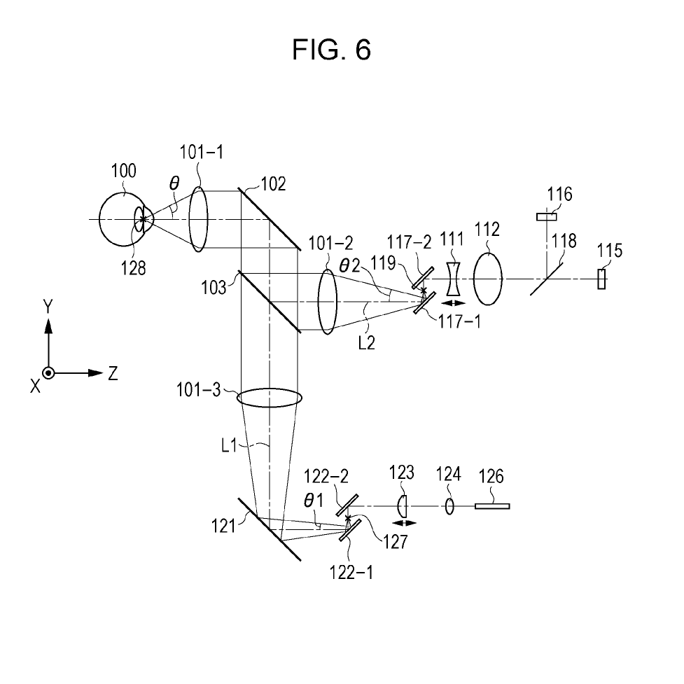

FIG. 6 illustrates the conjugate relationship of pupil position with regard to the optical path L1 and optical path L2, and the light flux of the pupil. The optical path L1 is the same as in the first embodiment, so description thereof will be omitted here. With regard to the optical path L2, a scanner center position 119 of the X-scanner 117-1 and Y-scanner 117-2, and an pupil position 128 of the eye 100 to be examined, are in a conjugate relationship. The lens 101-2 and the scanner center position 119 (X-scanner 117-1 and Y-scanner 117-2) are situated such that the light flux between the lens 101-1 and lens 101-2 is generally parallel. According to this configuration, the optical path of which a measurement light deflecting unit is an object point is generally parallel between the lens 101-1 and lens 101-2. Accordingly, the incident angle to the first dichroic mirror 102 and the second dichroic mirror 103 can be made to be the same regardless of having performed scanning at the X-scanner 117-1 and Y-scanner 117-2.

Also, the optical path L1 and optical path L2 are configured to share the lens 101-1, and further the lens 101-2 and lens 101-3 are configured using lenses of the same shape and same material. Thus, both the optical path L1 and optical path L2 can have the same optical system up to the respective X and Y scanners, and both optical paths can have the same optical characteristics.

As illustrated in FIG. 6, the angle of the light flux to the pupil as to the pupil of the eye 100 to be examined is θ, the angle of the light flux to the pupil as to the scanner center position 127 is θ1, and the angle of the light flux to the pupil as to the scanner center position 119 is θ2. That is to say, the beam is given the angles θ1 and θ2 using the scanners, so as to obtaining the angle θ of the light flux to the pupil on both optical paths L1 and L2.

As another optical characteristic, the optical magnification of the scanner center position 119 as to the pupil position 128, and the optical magnification of the scanner center position 127 as to the pupil position 128, can be made the same on either optical path. As a result, the relationship between the scan angles of the X and Y scanners on the respective optical paths and the irradiation position on the fundus of the eye 100 to be examined can be made equal between the two optical paths, so that θ1=θ2. Accordingly, scan position error of each optical path can be reduced.

As described above, the wavelength separation precision of the optical tomographic imaging apparatus according to the present embodiment can be improved by making the angle of incidence of the beams to the dichroic mirrors to be constant. Also, placing a focusing lens between the irradiation light source at the fiber end and the X and Y scanners enables the driving mechanism to be simplified. Further, the irradiation light source does not need to be moved, so a optical tomographic imaging apparatus with a stable polarization state can be provided. The same lenses are used on the OCT measurement optical path and the fundus observation optical path, whereby measurement error can be reduced.

### Third Embodiment

### Beam Flux

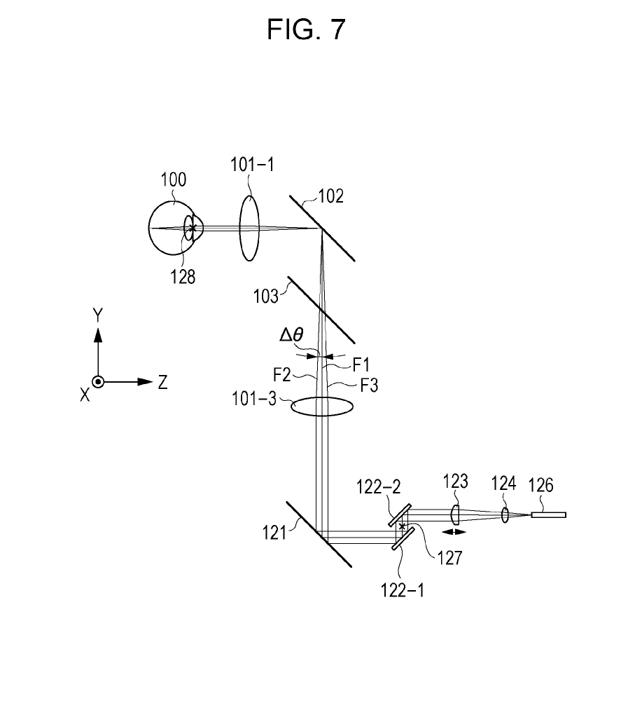

The configuration of an optical tomographic imaging apparatus (OCT apparatus) according to a third embodiment will be described with reference to FIG. 7. The OCT apparatus includes an optical head 900 and spectroscope 180, in the same way as the first embodiment. An optical system has been described in the first embodiment where a light flux which has passed through the first dichroic mirror 102 and second dichroic mirror 103 becomes generally parallel to the optical axis at an optional scan position. Conversely, in the third embodiment, the light flux has a finite spread, with the rays in the light flux not being in a mutually parallel relationship but rather having a spread angle. The configurations of the other optical paths L1 and L3, and the configuration of the spectroscope 180 are the same as described in the first embodiment, so description will be omitted here.

### About Spread of Light Flux

Light flux conditions, which is the difference as to the first embodiment, will primarily be described below.

FIG. 7 illustrates the light flux spreading when the X-scanner 122-1 and Y-scanner 122-2 are at center angles. F1, F2 and F3 represent light rays in the light flux. The light ray F3 is the principal ray of the light flux and matches the center of the optical axis of the optical path L1, and passes through the center of the X-scanner 122-1 and Y-scanner 122-2 and the pupil position 128. The light rays F2 and F3 are peripheral rays of the light flux, and are equivalent to a Gaussian beam radius which has 1/e² intensity of the peak in a Gaussian intensity distribution flux. Rays continuously exist on the range of peripheral rays F2 through F3 including the principal ray F1 in the light flux. Note that the peripheral rays F2 through F3 are not restricted to a Gaussian beam radius, and may be rays equivalent to an effective diameter according to an intensity taken into consideration in design.

### About Inclination of Rays

As illustrated in FIG. 7, the principal ray F1 is generally parallel to the optical axis at the regions of passing through the first dichroic mirror 102 and second dichroic mirror 103, according to the configuration illustrated in the first embodiment. On the other hand, the peripheral rays F2 and F3 have positive/negative symmetrical angles with respect to the optical axis. The term inclination as used here means the angle formed between peripheral rays F2 and F3 with respect to the optical axis. Rays in the light flux continuously have an inclination within a range of zero degrees (light parallel to the optical axis) and a maximum inclination being that of the peripheral rays F2 and F3 (greatest inclination). In FIG. 7, the greatest inclination is illustrated as the angle Δθ between F2 and F3. This angle changes depending on focal adjustment with regard to the eye to be examined. The greatest inclination is greater when the eye to be examined is more hyperopic, and is smaller when the eye to be examined is more myopic. The higher the greatest inclination is, the lower the wavelength separation accuracy is. Note that the relationship of inclination angle among the rays in the light flux is constant regardless of the scanning direction, so the uniformity of the tomographic image is maintained.

### About Parameters

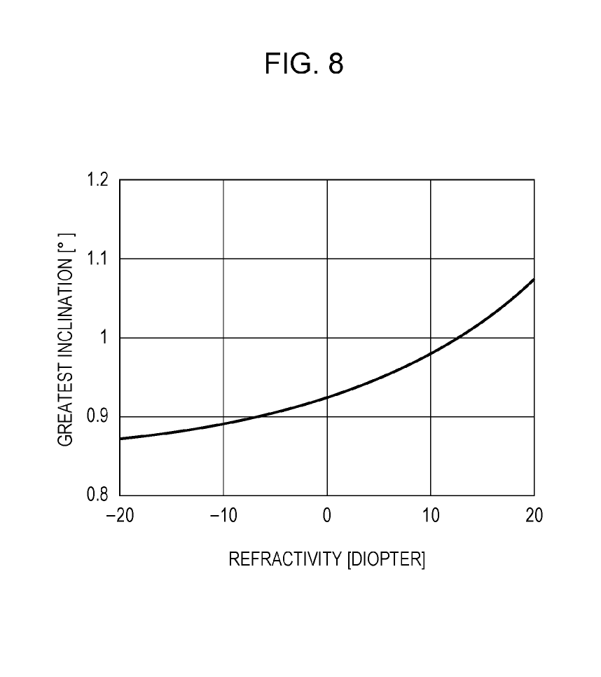

FIG. 8 is a graph illustrating the relationship in change of the greatest inclination in the light flux passing through the second dichroic mirror 103 as to the refractivity of the eye to be examined, where focal adjustment is performed. The axis of abscissae represents the refractivity [diopter] of the eye to be examined, and the axis of ordinates is the greatest inclination Δθ (in degrees). The light flux includes rays having inclinations in this range of ±Δθ. The greatest inclination as to each refractivity, Δθ(D), is obtained from Expression (1) shown below

Δθ(D)=NA/β(D) (1)

according to the numerical aperture (NA) of the measurement light source and primary imaging magnification in the OCT apparatus as to each refractivity, β(D). In FIG. 8, if the optical system is configured such that, with NA of 0.14 and primary imaging magnification β(D) of −18 diopters, 0 diopters, and +15 diopters, β(−18)=9.3, β(0)=8.8, and θ(15)=8.0, then Δθ(D) will respectively be Δθ(−18)=0.88, Δθ(0)=0.92, and Δθ(15)=1.02. The larger the refractivity of the eye to be examined is, the larger the greatest inclination tends to be, so at +15 diopters which is the greatest within the range of refractivity in which the OCT apparatus is capable of imaging, the value is around 1.02 degrees, as described above. While having Δθ as close to zero as possible is desirable regarding wavelength separation characteristics, reducing the numerical aperture NA to realize this markedly reduces beam output, while increasing the primary imaging magnification β greatly changes the overall length of the optical system, and further results in an enlarged spot size on the fundus, reducing the resolution of the optical image, and greatly reducing the functions of the OCT apparatus. About Tolerance Angle Range of Dichroic Mirror

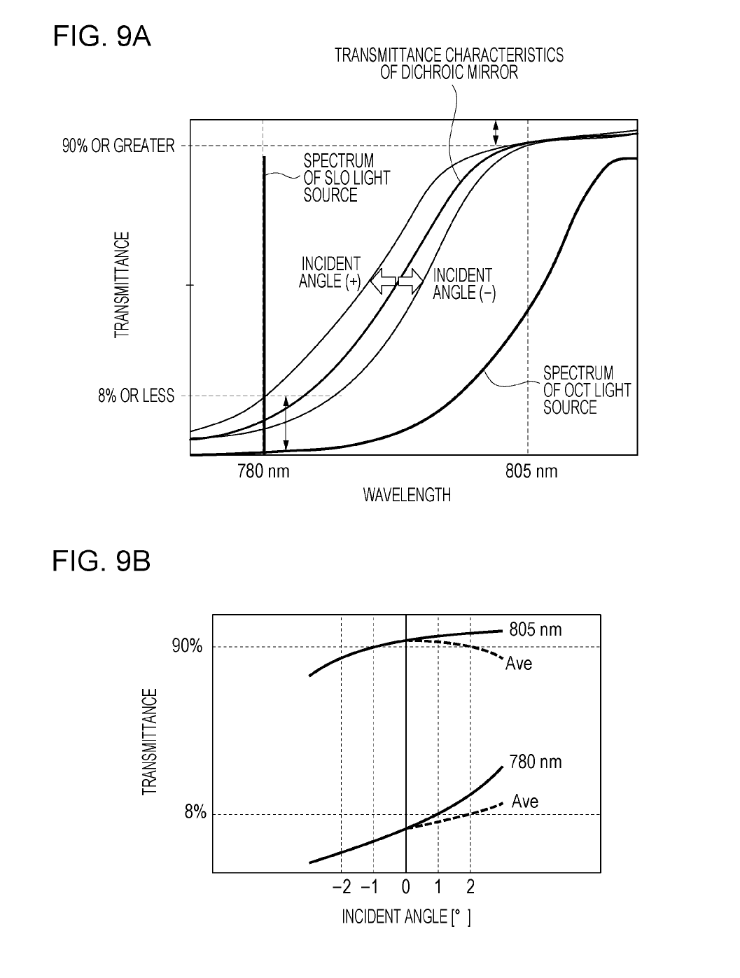

FIGS. 9A and 9B illustrate graphs of the relationship between the incident angle of measurement light rays and the transmittance properties of the second dichroic mirror 103. In FIG. 9A, the axis of abscissae represents wavelength (in nm), and the axis of ordinates represents transmittance. The transmittance properties of the second dichroic mirror, the separated OCT light source spectrum, and the SLO light source spectrum are plotted in FIG. 9A. The transmittance characteristics of the dichroic mirror are platted along with the shift in the curve in cases where the incident angle of light rays change in the positive or negative directions. If the incident angle changes toward the positive side, there is a shift toward the shorter wavelength side, and if the incident angle changes toward the negative side, there is a shift toward the longer wavelength side.

The OCT light source spectrum requires higher transmissivity, and transmissivity of 90% or higher is desirable at a wavelength of 805 nm. On the other hand, the SLO light source spectrum requires higher reflectance, i.e., lower transmissivity, with transmissivity of 8% or less being desirable. From this relation, in a case where light rays having a wide range of inclinations are included in the light flux, the above-described range of characteristics is preferably maintained even if transmittance characteristics do shift.

FIG. 9B is a graph where FIG. 9A has been reorganized, with the axis of abscissae representing shift of the incident angle of light rays as to 45 degrees, and the axis of ordinates represents the transmissivity of the second dichroic mirror 103. The solid lines represent transmissivity as to the incident angle at wavelengths of 805 nm and 780 nm, and monotonously increase as the incident angle increases. On the other hand, the broken lines denoted “Ave” are the respective transmissivity properties averaged over the positive and negative range. Averaging enables the overall transmissivity change within the range of maximum inclination of the light flux to be evaluated. Both transmissivity characteristics exhibit nonlinear relationships, with the curve peaking upwards in the case of the wavelength 805 nm and the curve peaking downwards in the case of the wavelength 780 nm. Accordingly, the average values are not constant values, but a value monotonously decreasing in the case of the wavelength 805 nm and a value monotonously increasing in the case of the wavelength 780 nm. From these characteristics, and the relationship in wavelength separation characteristics, it can be seen that the greatest inclination Δθ preferably is within, i.e., no greater than, 2 degrees.

### About Configuration Conditions of Optical System

From the relationships in FIGS. 8 through 9B, the primary imaging magnification β(D) of the optical system and the numerical aperture NA of the measurement light source (numerical aperture of the fiber) can be suitably decided following Expression (1), so that the greatest inclination Δθ falls within the range of Δθ 2 degrees on the optical path passing through the second dichroic mirror 103, within the range of refractivity in which the OCT apparatus is capable of imaging. One way to decide this is to reduce the NA and increase β(D), in which case, for example, the NA can be reduced by reducing the size of the measurement light source 126 which is the end of the fiber, and then adjust the output of the light source 130 to make up for the correspondingly lower output in accordance with the photoreceiving sensitivity of the line sensor 184. On the other hand, β(D) can be increased by shortening the focal distance of the lenses 123 and 124, or increasing the focal distance of the lenses 101-1 and 101-3, within the range of restrictions which are the overall length of the optical system, the resolution of focal adjustment, and optical resolution on the fundus. Such a configuration enables the second dichroic mirror 103 to exhibit good wavelength separation characteristics as to the OCT light source and SLO light source.

### Other Embodiments

Embodiments of the present invention can also be realized by a computer of a system or apparatus that reads out and executes computer executable instructions recorded on a storage medium (e.g., non-transitory computer-readable storage medium) to perform the functions of one or more of the above-described embodiment(s) of the present invention, and by a method performed by the computer of the system or apparatus by, for example, reading out and executing the computer executable instructions from the storage medium to perform the functions of one or more of the above-described embodiment(s). The computer may comprise one or more of a central processing unit (CPU), micro processing unit (MPU), or other circuitry, and may include a network of separate computers or separate computer processors. The computer executable instructions may be provided to the computer, for example, from a network or the storage medium. The storage medium may include, for example, one or more of a hard disk, a random-access memory (RAM), a read only memory (ROM), a storage of distributed computing systems, an optical disk (such as a compact disc (CD), digital versatile disc (DVD), or Blu-ray Disc (BD)™), a flash memory device, a memory card, and the like.

While the present invention has been described with reference to exemplary embodiments, it is to be understood that the invention is not limited to the disclosed exemplary embodiments. The scope of the following claims is to be accorded the broadest interpretation so as to encompass all such modifications and equivalent structures and functions.

This application claims the benefit of Japanese Patent Application No. 2013-173231 filed Aug. 23, 2013, which is hereby incorporated by reference herein in its entirety.

What is claimed is:

1. An optical tomographic imaging apparatus which acquires tomographic images of an object to be examined, where the images are based on multiplexed reference light and return light returned from the object to be examined which has been irradiated by measurement light, the apparatus comprising:
a first lens;
a scanning unit disposed on the optical path of the measurement light, and configured to scan with the measurement light the object to be examined;
a second lens disposed on the optical path of the measurement light between the scanning unit and the first lens;
an optical path branching unit disposed between the first lens and the second lens, and configured to branch, from the optical path of the measurement light, an observation optical path to observe the object to be examined;
a splitting unit configured to split light irradiated from a light source into the measurement light and the reference light; and
a focusing lens disposed on the optical path of the measurement light between the splitting unit and the scanning unit;
wherein the second lens and the scanning unit are disposed such that an incident angle of the measurement light, scanned by the scanning unit, to the optical path branching unit is maintained;
and wherein, on the optical path between the first lens and the second lens, a primary magnification of the optical system on the optical path of the measurement light or the observation light path, and a numerical aperture of the light source irradiating the measurement light, are configured such that a maximum inclination of light rays included in the light flux of the measurement light is maintained within ±2 degrees of inclination with respect to the optical axis of the optical path.

2. The optical tomographic imaging apparatus according to claim 1, further comprising:
a driving unit configured to drive the focusing lens along the optical path of the measurement light.

3. The optical tomographic imaging apparatus according to claim 1,
wherein the scanning unit further includes a first scanning unit which scans the object to be examined by the measurement light in a first direction, and a second scanning unit which scans in a second direction which intersects the first direction;
and wherein a position conjugate with a predetermined part of the object to be examined is disposed so as to be between the first and second scanning units.

4. The optical tomographic imaging apparatus according to claim 1, further comprising:
an optical fiber disposed on the optical path of the measurement light;
wherein the splitting unit is a photocoupler connected to the optical fiber;
and wherein the focusing lens is disposed between an end of the optical fiber and the scanning unit.

5. The optical tomographic imaging apparatus according to claim 1, wherein the first lens, the second lens, and the scanning unit are disposed so that light on the optical path of the measurement light between the first lens and the second lens is parallel light.

6. The optical tomographic imaging apparatus according to claim 5, further comprising:
an observation scanning unit configured to scan the object to be examined by observation light irradiated from an observation light source; and
a third lens, disposed on the observation optical path, between the second scanning unit and the object to be examined.

7. The optical tomographic imaging apparatus according to claim 6,
wherein the observation scanning unit further includes a first observation scanning unit configured to scan the object to be examined by the observation light in a first direction, and a second observation scanning unit configured to scan in a second direction intersecting the first direction;
and wherein a position conjugate with a predetermined part of the object to be examined is disposed so as to be between the first and second observation scanning units.

8. The optical tomographic imaging apparatus according to claim 6, wherein the first lens, the third lens, and the observation scanning unit are disposed so that light on the optical path of the observation light between the first lens and the third lens is parallel light.
